(19) United States
(12) Patent Application Publication
(10) Pub. No.: US 2003/0175342 A1
(43) Pub. Date: Sep. 18, 2003

(54) Coated pharmaceutical single-unit delayed-release forms, based on polyvinyl acetate

(76) Inventors: Karl Kolter, Limburgerhof (DE); Silke Gebert, Grunstadt (DE); Bernhard Fussnegger, Kirrweiler (DE); Anisul Quadir, Flanders, NJ (US)

Correspondence Address: KEIL & WEINKAUF, 1350 CONNECTICUT AVENUE, N.W., WASHINGTON, DC, 20036, US

(21) Appl. No.: 10/096,835

(22) Filed: Mar. 14, 2002

(51) Int. Cl. A61K 9/22; A61K 9/16; A61K 9/50

(52) U.S. Cl. 424/468

(57) ABSTRACT

The present invention relates to pharmaceutical single-unit administration forms provided with a sealed film coating and having delayed release, the film coating containing 30 to 100% by weight of polyvinyl acetate and the layer thickness of the film coating being 30 μm to 500 μm.

The present invention relates to pharmaceutical single-unit aministration forms provided with a sealed film coating and having delayed release, the film coating containing 30 to 100% by weight of polyvinyl acetate and the layer thickness of the film coating being 30 μm to 500 μm.

Furthermore, corresponding coating compositions and a process for the production of the coatings have been found.

In pharmaceutical delayed-release forms, a differentiation is made between “single unit” or “bolus” forms and “multiple unit” forms. A single unit form consists only of a sole molded body, whereas the multiple unit forms are composed of a large number of small molded articles. Single unit forms are as a rule simpler to prepare, especially since the multiple unit forms need a further packaging unit such as a capsule shell. Examples of single unit forms are tablets or extrudates; pellets, granules or crystals are employed as multiple unit forms.

On account of the release-delaying principle, a division into coated delayed-release forms and matrix forms can moreover additionally be made. The coated forms bear a coating which slows diffusion and thus release. In the matrix forms, the active compound is present dispersed in a coherent matrix, where the matrix can consist of water-insoluble or water-swelling materials. Since the diffusion route of the active compound out of the matrix form as a rule becomes longer with time, the release rate also slows as a function of time.

Since the delayed-release forms carry a larger amount of pharmaceutical than rapid-release forms, which instead have to be taken a number of times in the day, the stability of the release of active compound is of enormous importance. In no case must dose dumping occur, because a large amount of pharmaceutical is released in a short time thereby and can lead to overdosage symptoms and significant side effects. This danger must also be excluded in the case of mechanical loading of the administration form, such as can occur during production, packaging or handling by the patient. In matrix forms, this danger is reduced by the uniform embedding of the pharmaceutical in the matrix. Coating forms, however, are significantly more sensitive to mechanical loading on account of their construction. In principle, even a small leak or a small crack suffices to release the entire dose in a short time. There are therefore almost no coated multiple unit delayed-release forms and no single unit delayed-release forms on the pharmaceutical market, since owing to the large number of molded articles the damage to individual molded articles only has a small effect on the overall release. It is easily imaginable that the damage to, for example, 5 molded articles of a total of 100 accelerates the release only slightly, namely by 5%. Damage to a coated single unit form would, however, lead to a 100% release in a short time. Furthermore, it is obvious that due to the higher weight of a single unit form and the higher kinetic energy on agitation, such as, for example, on shaking, caused thereby, the risk of damage is markedly greater than with multiple unit forms.

A particular form of release delaying is the “OROS system”. In this, a core, which is osmotically active, is surrounded with a very rigid shell which is permeable to water, but not to the pharmaceutical. In this coating, with the aid of a laser, a hole of a defined size is burnt, through which the pharmaceutical or the pharmaceutical solution or suspension formed after water has diffused in can escape at a specific rate. The rate of release depends on the osmotic activity of the core, the permeability of the coating to water and on the size of the hole. This pharmaceutical form has numerous disadvantages; thus its preparation is very complicated and expensive, since a defined hole has to be burnt into each individual tablet. During this burning process, polymeric parts are thermally decomposed, whereby undefined and toxicologically hazardous compounds can result. The coating must be very thick and rigid and can only be applied from organic solution. In order to be suitably active osmotically, salts must be incorporated into the core, which in some cases are physiologically not particularly tolerable and reduce the pharmaceutical concentration in the core. The size of the administration form increases as a result.

Typical OROS system [sic] are described, for example, in DE 19747261 A1 and EP 0277092 B1. The disadvantages and limitations are as explained above.

An OROS system which can be used for colon targeting is mentioned in U.S. Pat. No. 4,904,474. With a number of layers and compartments, production is extremely laborious and complex and organic solvents have to be employed.

In GB 2267219 A, coated tablets containing active compounds against coughs and colds, which are situated in the core and in the shell, are described. The coating consists of a nonpermeable component such as ethyl cellulose, cellulose acetate or polycarbonate and a permeable component such as polyvinylpyrrolidone, fumaric acid, citric acid, sodium citrate, monosaccharides or disaccharides. Polyvinyl acetate or an aqueous dipsersion of polyvinyl acetate is not mentioned. On account of the polymer composition, this coating is brittle and thus susceptible to mechanical loading and it must be applied from organic solution (methylated spirits). From the ecological point of view and on account of the higher risk potential, use of organic solvents is not sensible. Moreover, it exhibits a strong curing effect, because the coated tablets have to be stored at 104 to 122° F. for 8 hours in order to adjust the release. This requires an additional outlay in terms of time and personnel and complicates the production process. The tablets furthermore carry a “cosmetic top coating” which in turn confirms that the underlying film controlling the diffusion is unattractive, because otherwise this top coating could be dispensed with. It makes production more expensive and complicates it further.

In EP 0211991 B1, delayed-release tablets are described which consists a coating [sic], consisting of a terpolymer of vinyl chloride (80 to 95%), vinyl acetate (1 to 19%) and vinyl alcohol (1 to 10%), and a water-soluble pore-forming substance. Vinyl chloride is not very suitble as a constituent of polymers on account of the toxicity for pharmaceutical products. Residual amounts of monomer always remain in the polymer, which can lead to corresponding side effects. A terpolymer is very difficult to prepare reproducibly, since the individual components react with differing ease and rapidity and are not always statistically distributed. Thus a reproducible release adjustment is not possible. The coating must be applied from an organic solvent (acetone, methylene chloride, methyl ethyl ketone), in which the terpolymer is dissolved. The mechanical properties of the coating are inadequate, because very large amounts of a nonfilm-forming or poorly film-forming substance such as sucrose are incorporated. The ratio of sucrose to terpolymer is 1 to 5, preferably 1.5 to 3. Comparatively high amounts of plasticizers are additionally added, which are generally undesirable, because plasticizers lead to changes in the administration form on storage on account of their volatility and their diffisuion power. The release is in particular affected thereby. The same applies for U.S. Pat. No. 4,557,925.

EP 0173928 B1 describes a similar film coating to EP 0211991 B1, however with the difference that the water-soluble, pore-forming substance consists of a pharmaceutical. Disadvantages and problems are the same as described above.

In U.S. Pat. No. 4,218,433, coated tablets are described which bear an indentation having a certain size such that the indentation is not coated during the coating process. Release takes place via this noncoated indentation. This system has a certain similarity to the OROS system, since here too water can permeate through the coating and the release of the pharmaceutical takes place through an opening in the coating. The edges of this indentation are very sensitive to shear, pressure and rolling stresses, as a result of which the release changes. The coating must be applied from organic solvents.

U.S. Pat. No. 4,252,786 and U.S. Pat. No. 4,610,870 describe coated tablets in which, by incorporation of a combination of polyvinylpyrrolidone and polyacrylic acid or swelling agents into the core, release is delayed. In addition, on this core is situated a coating which consists of a hydrophobic polymer and a hydrophilic polymer and which splits open or erodes after a certain time. It is intended only to reduce the initial release. This coating thus has very different objects and properties than a pure retard coating, which must remain intact over the entire release period.

The coating in EP 0282011 A2 performs a similar function, namely only a reduction of the initial relase of a matrix delayed-release form. The difference in the release between coated and noncoated form is low.

In U.S. Pat. No. 5,458,887, an osmotically active core which contains a swelling agent (either hydroxypropylmethylcellulose or polyethylene oxide) is covered with a coating of a hydrophobic polymer and a water-soluble, low molecular weight, mostly inorganic compound. The properties of the coating are inadequate, since the hydrophobic polymers do not have the necessary flexibility and the low molecular weight addition worsens the mechanical properties. Moreover, organic solvents and a very high plasticizer content are employed.

Coatings which split open or become permeable after a certain time are also described in WO 00/78293 and EP 0408496 A2.

In EP 0335560 B1, controlled release administration forms are described which carry a porous film having a porosity of 0.4 to 0.9, which has been applied from an organic solvent. It is evident that such porous films are not stable mechanically. On the contrary, it is to be desired that the films have a very low porosity.

The films described in U.S. Pat. No. 4,572,833, in addition to a controlled release polymers, contain a molten, hydrophobic substance which is insoluble in the organic solvent used. The production of the coating is very laborious, since the preparation has to be warmed and highly dispersed to melt the hydrophobic substance (e.g. wax). The film properties and also the adhesion of the film to the tablet surface are considerably worsened by the content of the hydrophobic substance.

EP 0655240 describes tablets coated with hydrophobic polymers and having slow release, where the active compounds must have a low water solubility of less than 5 mg/ml. As hydrophobic polymers, ethyl cellulose and acrylate-methacrylate copolymers are mentioned. Polyvinyl acetate is not mentioned in any manner. These formulations require a high plasticizer content and, after production, must be laboriously tempered until the release is constant and are mechanically unstable.

In U.S. Pat. No. 4,871,546, film-coated tablets are described which carry an insoluble coating which is intended to protect the gastric mucous membrane from irritating pharmaceuticals. The coating consists of a combination of polymethyl methacrylate, polyvinyl acetate and polyethylene glycol and must be applied from organic solution. The concentration of these polymers in the organic solution is low for viscosity reasons, as a result of which very long coating times and high costs result. The release is complete after 2 to 4 h, i.e. what is concerned here is not a delayed-release coating. Moreover, the coating is poorly reproducible, since large release differences occur between individual tablets.

In WO 97/02020, specific pantoprazole formulations are described which carry a two-layer coating, a hydrophobic polymer being employed for the intermediate layer. This film coating is, however, stabilized by a further layer, consisting of an enteric polymer. The preparation is very laborious and moreover on account of the enteric polymer no release takes place in the gastric juice.

U.S. Pat. No. 4,756,911 describes coated tablets of procainamide, the core being retarded using a hydrocolloid and/or a wax and the coating consisting of a hydrophilic hydroxypropylmethylcellulose and a lipophilic polymer (ethylcellulose). The coating is in no manner stable, especially not in the stomach and intestinal tract because it is described that the coating peels off after 2 to 4 h in the gastric/intestinal juice. It thus brings about no specific release control. Moreover, it is applied in a laborious and ecologically unadvantageous manner from organic solution. Polyvinyl acetate is not mentioned in this patent.

In U.S. Pat. No. 5,175,003, oral pharmaceuticals are disclosed which are provided with a pH-dependent film coating, the film coating consisting of a pH-dependent soluble polymeric coating agent and a film-forming agent having a plastifying action.

In EP-A 438 249, naproxen-containing administration forms having controlled release are disclosed in which an active compound-containing pellet core is provided with a multilayer coating, comprising a water-insoluble film-forming agent.

In WO 99/01129, preparations of dihydropyridine calcium antagonists are disclosed in which initially a pH-independent coating is applied to microgranules coated with active compound, and subsequently further hydrophilic or lipophilic coating layers are applied.

In WO 01/15668, pellet formulations having controlled release are disclosed which are provided with a coating of a water-insoluble polymer and a pH-dependent soluble polymer.

In U.S. Pat. No. 5,202,128, pellet formulations having controlled release are disclosed in which a core which contains a readily water-soluble active compound are [sic] coated with a film coating which consists of a number of different pH-dependent soluble polymers.

In EP-A 868912, coating materials consisting of 10 to 95% by weight of polyvinyl acetate and 5 to 90% by weight of an N-vinylpyrrolidone-containing polymer are disclosed.

In EP-A 1110544, the use of a film coating consisting of polyvinyl acetate and hydrophilic additives as taste-masking coating for oral administration forms is disclosed, the forms being essentially of rapid-release type.

It has hitherto not been possible to find a coating which can be applied to a core by means of a simple aqueous film-coating agent and which has such good mechanical properties that no dose dumping occurs. In particular, it is not possible according to the prior art to provide flexible molded articles such as capsules having a coating which complies with the requirements mentioned.

It is an object of the present invention to find improved film coatings which do not have the disadvantages outlined. We have found that this object is achieved by the administration forms defined at the outset.

Single-unit forms are designated as monolithic pharmaceutical forms a number of millimeters in size (>5 mm).

The present invention describes a release-delaying film coating which has appropriate mechanical strengths so that the customary mechanical stresses of the coated molded article lead to no change in release whatsoever. This presupposes that the film coating has a high elasticity and withstands pressure and shear stresses. In no case must the coating be brittle, because otherwise it would develop cracks during expansion of the core. The coating according to the invention moreover has a “self repair mechanism”, i.e. damage to the coating is repaired by the plastic behavior. Thus the coating can be pricked, for example, with a needle and the administration form in spite of this shows no change in the release compared with an undamaged form. The explanation for this unexpected and surprising behavior is probably based in the plastic properties of the coating. In the presence of water or aqueous media, leaks are sealed by flowing together of the edges of the leak. As a result, the release which occurs is the same as without damage. This behavior is unique and specific for polyvinyl acetate.

The coatings according to the invention thus offer double protection from dose dumping and thus considerably increase pharmaceutical safety:

1. owing to the high flexibility they resist mechanical stresses

2. if damage does occur, this is even repaired

The properties of the coatings according to the invention mentioned are all the more surprising, since they are achieved from an aqueous base and it is clear to any person skilled in the art that the achievement of good film properties from an aqueous base is essentially more difficult than using organic solvents. Many pharmaceuticals and also the OROS systems are therefore prepared by means of organic coating. In the case of the use of aqueous systems, the polymer particles must be constituted such that they flow together very readily and afford a homogeneous film which is stable on storage, but are [sic] also not sticky on the other side. It has been found that only polyvinyl acetate has these desired properties. Other polymers are not suitable or less suitable for this purpose.

The coatings according to the invention moreover have the following advantages:

simple application (simple coating process)

high spray concentrations

no stickiness

excellent smoothness and gloss

high pigment-binding power

low production costs

high storage stability

low plasticizer concentration.

In contrast to the OROS system, the release of active compound does not take place locally by escape through a hole in the coating, but uniformly by diffusion through the entire film surface. High local pharmaceutical concentrations, which can lead to damage to the gastric or intestinal mucous membrane, are thereby avoided. The systems according to the invention are thus markedly more tolerable than OROS systems.

Compared with the prior art, the administration forms according to the invention thus offer a considerable advance, they really allow for the first time the simple and safe production and use of a coated single unit administration form.

For the preparation of the administration forms or coatings according to the invention, the use of polyvinyl acetate is essential, since only this polymer has the plastic/elastic properties mentioned. It is preferably employed in the form of an aqueous dispersion. The average molecular weight should be between 10000 and 2000000, preferably between 100000 and 1000000.

The content of the polyvinyl acetate in the coating layer is 30 to 100% by weight.

The release of the active compound can be adjusted by means of the layer thickness of the coating layer and by means of the addition of water-soluble or water-swellable substances. The water-soluble substances are dissolved out of the film to a greater or lesser extent in the gastric or intestinal juice, as a result of which the permeation of the active compound increases.

The water-soluble substances employed can be all sorts of compound classes, but polymers have proven particularly suitable, since these advantageously act on the film properties.

Particularly suitable polymers are: polyvinyl alcohols, polyvinyl alcohol-polyethylene glycol graft copolymers, polyethylene glykols, ethylene oxide-propylene oxide block copolymers, alkylated celluloses such as, for example, methyl cellulose, hydroxyalkylated celluloses such as, for example, hydroxypropylcellulose, hydroxypropylmethylcellulose or hydroxyethylcellulose, alkylated-hydroxyalkylated celluloses, carboxyalkylated celluloses such as, for example, carboxymethylcellulose, polyvinylpyrrolidones, polyacrylates, polymethacrylates, acrylate-methacrylate copolymers, polysaccharides such as, for example, xanthan, galactomannans, dextrans, inulin, polydextrose, maltodextrins, alginates, propylene glycol alginates, chitosans, carrageenans, gum arabic, including their salts or derivatives.

The water-swellable substances create corresponding zones having higher active compound permeability in the film by the absorption of water, as a result of which the release likewise increases. Suitable water-swellable polymers are: crosslinked polyvinylpyrrolidones, crosslinked polyacrylic acid, microcrystalline cellulose, starch, crosslinked sodium carboxymethylstarch, crosslinked sodium carboxymethylcellulose, pectins. Care is to be taken here, however, that the water-swellable substances are not employed in such amounts that they act as disintegrants.

The water-swellable polymers must be employed in fine form in order that the film structure is not affected.

In principle, low molecular weight water-soluble substances can also be used. These include, for example, sugars such as sucrose, glucose, lactose, sorbitol, mannitol, isomalt, xylitol, sugar derivatives, urea, salts of organic or inorganic acids such as, for example, sodium citrate, sodium dihydrogen phosphate, sodium chloride.

The amounts of water-soluble or water-swellable substances in the film coating are between 2 and 50% by weight, preferably between 5 and 30% by weight.

The elasticity of polyvinyl acetate or of the entire coating can be further increased by small additions of plasticizers. Suitable plasticizers are in principle all substances which have a certain dissolving power for polyvinyl acetate and a boiling point above 100° C. Propylene glycol, glycerol, triethyl citrate, tributyl citrate, triacetin, acetyl tributyl citrate, diethyl phthalate, dibutyl phthalate, dibutyl sebacate, polyethylene glycols, glycerol monostearate, for example, are suitable. Triacetin, triethyl citrate and propylene glycol are particularly suitable. The concentration of the plasticizer relative to the film-forming agent should be between [sic] 0.5 to 25%, preferably 1 to 15% by weight, particularly preferably 3 to 10%.

Moreover, the administration forms according to the invention can contain further customary additives such as

colorants in water-soluble or water-insoluble form, e.g. iron oxides, Quinoline Yellow, erythrosine, indigo carmine, Brillant Black, beta-carotene, azorubin lake, indigotine lake, cochineal lake, Quinoline Yellow lake

white pigments for increasing the covering power of the coating, e.g. titanium dioxide, talc

antiadhesive agents, e.g. talc, magnesium stearate, glycerol monostearate, silicic acid

fillers, e.g. sucrose, glucose, lactose, sorbitol, mannitol, isomalt, xylitol, calcium phosphates

gloss intensifiers, e.g. waxes, fatty alcohol or fatty acid derivatives, polyethylene glycols

surfactants for improving the wetting behavior and spreading, e.g. sodium lauryl sulfate, Cremophor RH 40, polysorbate 80, sodium dioctyl sulfosuccinate

foam destroyers

The amounts of these substances which are customary for pharmaceutical film coatings including plasticizer in the film coating can be up to 40% by weight.

The excellent elastic-plastic properties of the polyvinyl acetate moreover allow the combination with other water-insoluble release-delaying polymers. The relation of these substances must, however, be above a ratio of polyvinyl acetate to further, water-insoluble release-delaying polymer of 1:1 in order that the properties of the polyvinyl acetate dominate. The further water-insoluble release-delaying polymers are best added in the form of an aqueous dispersion. Alkylated polysaccharides such as ethyl cellulose or acrylate-methacrylate copolymers such as methyl methacrylate-ethyl acrylate copolymer, ammonium methacrylate copolymer type A or B, for example, are suitable. The amount of these delayed-release polymers in the coating can be up to 40% by weight.

The rate of the release of active compound can be adjusted via the layer thickness of the film coating. It is to be taken into consideration here that a minimum layer thickness of about 10 μm is absolutely necessary in order to guarantee the mechanical stability. Suitable layer thicknesses are between [sic] 10 to 500 μm, preferably between [sic] 30 to 300 μm. In principle, a very readily water-soluble pharmaceutical needs a higher layer thickness in order to produce a certain release profile than a poorly water-soluble one.

The film coating can consist of a number of layers which differ with respect to their composition. Thus, for example, the content of polyvinyl acetate in the layers can be chosen differently. A high content of polyvinyl acetate combined with a low content of water-soluble or water-swellable substances leads to a low permeability for pharmaceuticals and a low content of polyvinyl acetate combined with a higher content of water-soluble or water-swellable substances leads to a high permeability for pharmaceuticals. In this manner, layers having different permeabilities can be combined in the coating. Moreover, it can be advantageous to concentrate color- or gloss-producing excipients in the outer layer, since they do not act in the interior.

Since initially water must permeate through the coating layer and the active compound must dissolve, in the initial phase of the release determination somewhat less active compound is released. This “lag phase” can be reduced or entirely eliminated by incorporating some of the active compound in the coating layer. The active compound then dissolves out of the layer relatively rapidly and produces a certain initial release.

As a rule, the course of release in the middle range, i.e. between 20 and 80%, is linear. Over 80%, it flattens as a rule, since the concentration of the dissolved active compound in the core decreases.

The production of the aqueous coating preparation to be sprayed takes place in a customary manner.

The application of the coating can be carried out in all coating devices suitable for tablets, such as, for example, horizontal drum coaters, fluidized bed coaters, immersed blade coaters, coating vessels.

For the atomization of the coating preparation, a two-substance nozzle is preferably used. The feed air temperature should be between 30 from [sic] 90° C., preferably between 40 to [sic] 80° C.

As a rule, after the application of the coating curing (tempering) is not necessary, since the film-forming properties of polyvinyl acetate are excellent. In isolated cases, in particular if higher contents of further coating constituents are incorporated, tempering of the coated molded articles at 30 to 70° C. for 0.2 to 24 h can stabilize the film properties, in particular the active compound release.

In principle, all core forms having a domed convex or concave surface are coated independently of whether they are round, polygonal, oblong or football-shaped forms. The molded articles can be produced by pressing processes, melt processes, casting processes, stamping processes, extrusion processes or injection molding processes. Therefore tablets, extrudates and forms which are cast, injection-molded, stamped or produced by melting and cooling can be employed for coating.

In particular, the coating is suitable for coating pharmaceutical capsules, because these are very flexible and require a particularly flexible coating. The coating must be as flexible as the capsule, otherwise damage in the coating results due to the unavoidable mechanical stresses and the delayed release action is adversely affected. Hitherto, it was not possible to delay the release of capsules safely and reproducibly by means of a coating. Of course, soft capsules are particularly suitable for the application according to the invention, but hard capsules can also be correspondingly coated, in particular after sealing the opening between the upper and lower part. The capsules can consist of different materials such as, for example, gelatin, starch, hydroxypropylmethylcellulose, polyvinyl alcohols. The core can also carry a subcoating, which as a rule is applied particularly in order to protect the pharmaceutical, e.g. from water, oxygen, protons or chemical substances of the coating and the gastric and intestinal contents.

Moreover, a further coating can be applied to the administration form coated according to the invention in order to achieve certain effects. On application of an enteric coating, no release would take place in the stomach, but a delayed release would take place in the small intestine and/or large intestine.

With respect to the active compounds, there are no restrictions for the administration forms according to the invention. Active compounds of all indication areas can be employed, human pharmaceuticals such as [sic] veterinary pharmaceuticals, vitamins, carotenoids, nutraceuticals, food supplements, minerals, micronutrients etc. The active compounds can have different physicochemical properties such as lipophilicity, solubility, grain size, grain structure, surface area, etc.

The active compound content of the core can be varied within wide limits. Thus cores having a low active compound content and also cores having very high active compound content can be used for the coating. The present invention is particularly advantageous in the case of very high-dose pharmaceuticals, since a tablet can be coated with up to 99.5% by weight of pharmaceutical, as a result of which the volume of the delayed-release form is very small and this is thus easily swallowable. However, low-dose pharmaceuticals can also be processed without problems to give the administration forms according to the invention, in which the core is increased to a convenient size using customary tablet fillers and excipients.

On account of the extraordinary flexibility of the coating, there are no restrictions for the composition of the core. All pharmaceutically customary excipients can be employed. Swelling excipients can also be used, since on account of the elasticity of the coating no crack formation occurs. Moreover, a slight retardation can also even be present in the core due to the incorporation of a release-delaying agent. The final retardation is then adjusted by means of the coating according to the invention. Possible release-delaying agents for the core are: waxes, fatty acids, modified fatty acids, fatty alcohols, modified fatty alcohols, modified fats, ethyl cellulose, polyvinyl acetate, polyvinyl acetate-polyvinylpyrrolidone, acrylate-methacrylate copolymers, methyl methacrylate-ethyl acrylate copolymer, ammonium methacrylate copolymer type A or B.

Incorporation is carried out either by dry intermixing together with the further recipe constituents or by granulation of the active compound with aqueous dispersions or suspensions of the water-insoluble lipids or polymers or with solutions of the lipids or polymers in organic solvents. The dry and the aqueous processing processes, of course, are preferred. In addition, the flowability of the powder mixture is improved by the granulation, so that better tableting results are achieved, or in cases of very poorly-flowing powders the tableting is first made possible at all.

The use of polymers of the same chemical structure as the coating is particularly preferred, namely based on polyvinyl acetate. Thus a polyvinyl acetate-polyvinylpyrrolidone blend can be employed for the dry intermixing and a polyvinyl acetate dispersion for the granulation. Incompatibilities and interactions between core and coating are thus almost excluded.

The flexibility of the coating is afforded not only in the dry state, but also in the moist state. After wetting with aqueous media, even the flexibility also markedly increases. Therefore no damage to the coating occurs even in the body as a result of the shear and pressure stress of the gastric and intestinal peristalsis.

The administration forms according to the invention can surprisingly be damaged, e.g. by pricks or cuts, without this having effects on the release. The plastic properties of the coating guarantee that in the gastric or intestinal tract the edges of the injured site slightly swell and flow together again as a result of the aqueous medium. Thus no dose dumping occurs even on damage to the coating. The coatings according to the invention are thus equipped with a self-repair mechanism.

The coated administration forms are so stable that they even withstand violent rolling and shaking movements without change in their properties. They can thus be subjected to friability testing in a Friabilator, where they fall downward over a distance of 15.5 cm 100 to 1000 times in a drum. In addition, the coated administration forms were allowed to fall 10-50 times from a falling height of 1 m onto a hard ground and then the properties, in particular the release, were determined. No difference could be found in this case. If, however, polyvinyl acetate is exchanged for other polymers, the abovementioned stresses immediately lead to dramatic accelerations in release. This shows the surprising, unique properties of polyvinyl acetate.

The coated bolus forms exhibit an excellent mechanical stability, have an exactly controllable, storage-stable release and show a self-repair mechanism, as a result of which dose dumping is prevented. The application of the film coating takes place without organic solvents.

### EXAMPLES

All percentages relate, if not stated otherwise, to percentages by weight.

### Example 1

### Metoprolol Delayed-Release Film-Coated Tablet

5 kg of rapid-release metoprolol tablets having a domed shape, a diameter of 9 mm and the following composition

| | |
|---|---|
| Metoprolol tartrate | 200.0 mg |
| Polyvinylpyrrolidone K30 | 6.0 mg |
| Di-Tab 1) | 160.0 mg |
| Aerosil 200 2) | 3.0 mg |
| Talc | 18.5 mg |
| Magnesium stearate | 4.0 mg |
| Total weight | 391.5 mg |

are sprayed in a horizontal drum coater (Accela Cota 24″, Manesty) with the following coating preparation:

| Kollicoat SR 30 D | 43.5% (corresponds to 13.05% |
|---|---|
| (30% strength aqueous | of solid) |
| polyvinyl acetate | |
| dispersion) | |
| Kollicoat IR 3) | 3.3% |
| Polyvinylpyrrolidone K30 | 0.5% |
| Titanium dioxide | 0.5% |
| Sicovit red (iron oxide) | 0.5% |
| Talc | 3.5% |
| Water | 48.2% |

The feed air temperature was 50° C., the product temperature 35° C., spray rate 25 g/min and the spray pressure 2.0 bar using a spray nozzle with a 1 mm opening width. The application rates were increased from 4 mg/cm² through 6 and 8 up to 10 mg/cm². The corresponding layer thicknesses were 38, 60, 78 and 100 μm.

The coated tablets were initially release for 2 h in 0.08 N HCl and then in phosphate buffer pH 6.8, the following active compound releases being achieved:

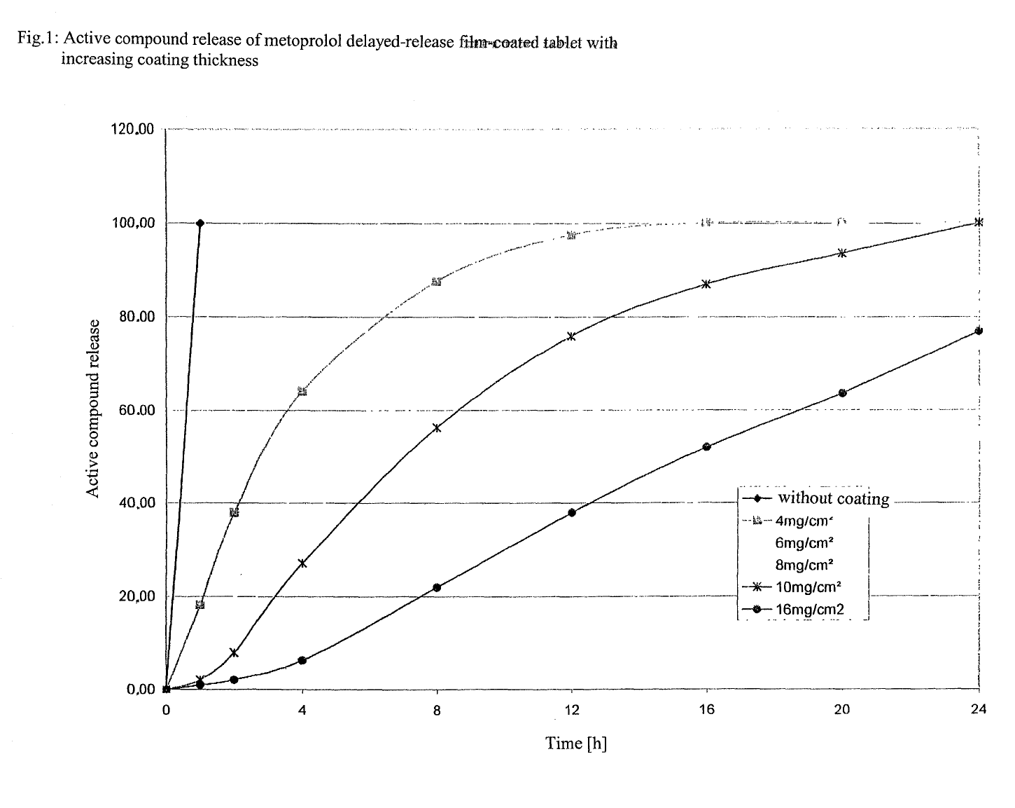

In FIG. 1, the active compound release from a metoprolol-containing delayed-release film-coated tablet having increasing layer thickness of the film coating is shown.

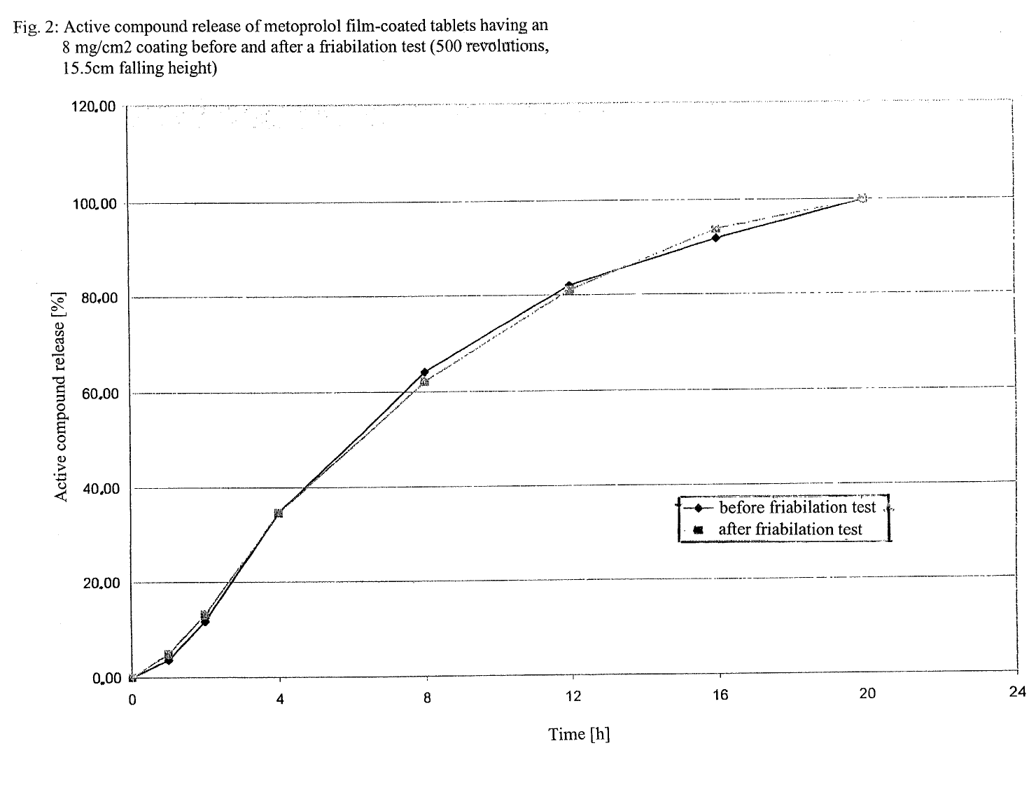

In FIG. 2, the active compound release of metoprolol film-coated tablets having an 8 mg/cm² film coating before and after a friabilation test (500 revolutions, 15.5 cm falling height) is shown.

The mechanical stress on the tablets produced no change in the release.

### Example 2

### Metoprolol Delayed-Release Film-Coated Tablet

5 kg of rapid-release metoprolol tablets having a domed shape, a diameter of 9 mm and the following composition

| | |
|---|---|
| Metoprolol tartrate | 200.0 mg |
| Polyvinylpyrrolidone K30 | 6.0 mg |
| Sorbitol | 160.0 mg |
| Aerosil 200 | 3.0 mg |
| Talc | 18.5 mg |
| Magnesium stearate | 4.0 mg |
| Total weight | 391.5 mg |

are sprayed in a horizontal drum coater (Accela Cota 24″, Manesty) with the following coating preparation:

| Kollicoat SR 30 D | 43.5% (corresponds to 13.05% |
|---|---|
| (30% strength aqueous | of solid) |
| polyvinyl acetate | |
| dispersion) | |
| Propylene glycol | 1.3% |
| Avicel PH 105 4) | 2.0% |
| Polyvinylpyrrolidone K30 | 0.5% |
| Titanium dioxide | 0.5% |
| Sicovit red (iron oxide) | 0.5% |
| Talc | 3.5% |
| Water | 48.2% |

The feed air temperature was 55° C., the product temperature 39° C., spray rate 30 g/min and the spray pressure 2.0 bar using a spray nozzle with a 1 mm opening width. 1125 g corresponding to an application rate of 8 mg/cm² were applied. The layer thickness was 82 μm.

The coated tablets were released in 0.08 N HCl (0 to 2 h) and then in phosphate buffer pH 6.8 (2 to 24 h). Furthermore, a hole was pierced in some of the coated tablets using a needle and some others were treated in the Friabilator (500 revolutions 15.5 cm falling height). The releases were unchanged.

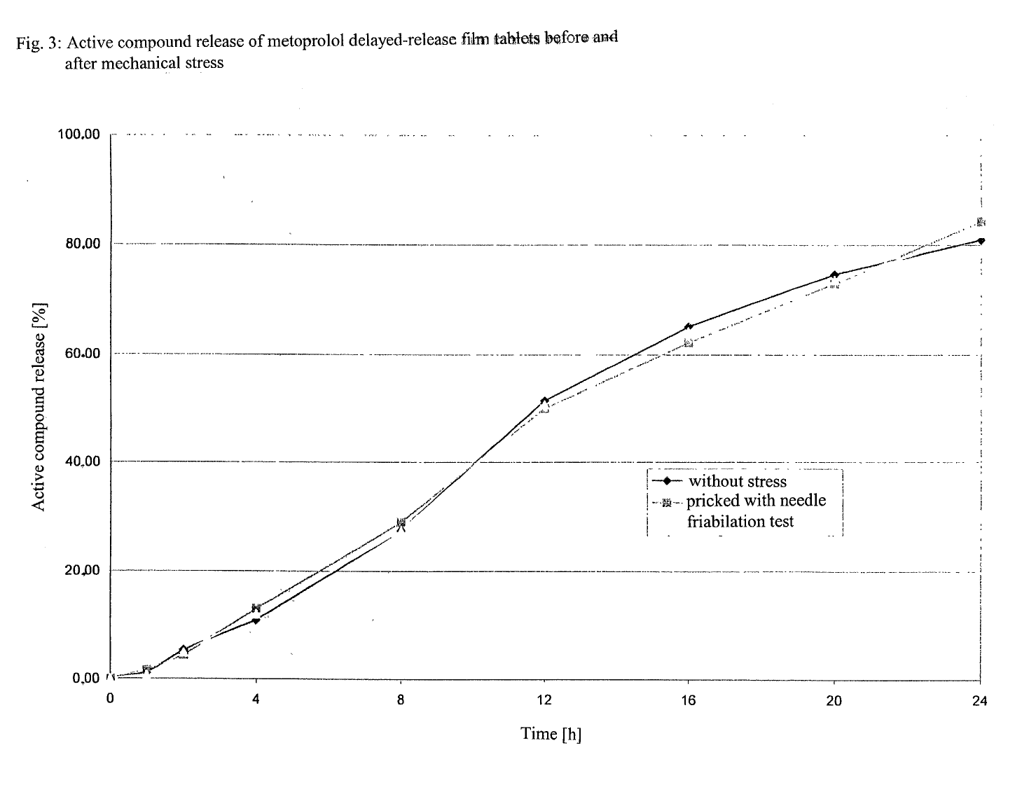

In FIG. 3 the active compound release of metoprolol delayed-release film-coated tablets before and after mechanical stress is shown

### Example 3

### Propranolol Delayed-Release Film-Coated Tablets

5 kg of rapid-release propranolol tablets having a domed shape, a diameter of 9 mm and the following composition

| | |
|---|---|
| Propranolol-HCl | 160.0 mg |
| Ludipress 5) | 140.0 mg |
| Crospovidone | 20.0 mg |
| Aerosil 200 | 3.0 mg |
| Magnesium stearate | 2.0 mg |
| Total weight | 325.0 mg |

were sprayed in a horizontal drum coater (Accela Cota 24″, Manesty) with the following film-coating preparation:

| Kollicoat SR 30 D | 43.5% (corresponds to 13.05% |
|---|---|
| (30% strength aqueous | of solid) |
| polyvinyl acetate | |
| dispersion) | |
| Propylene glycol | 2.0% |
| Avicel PU 105 6) | 2.0% |
| Kollidon VA 64 7) | 3.0% |
| Talc | 3.5% |
| Water | 45.0% |

The feed air temperature was 60° C., the product temperature 41° C. and the spray pressure 2.0 bar using a spray nozzle with a 1 mm opening width. The application rate was 16 mg/cm² corresponding to an amount of coating suspension of 2404 g. The layer thickness was 155 μm.

The coated tablets were released in 0.08 N HCl (0 to 2 h) and then in phosphate buffer pH 6.8 (2 to 24 h). Furthermore, a cut about 2 mm long was made in some of the coated tablets using a razor blade and the release was likewise determined. The releases were unchanged.

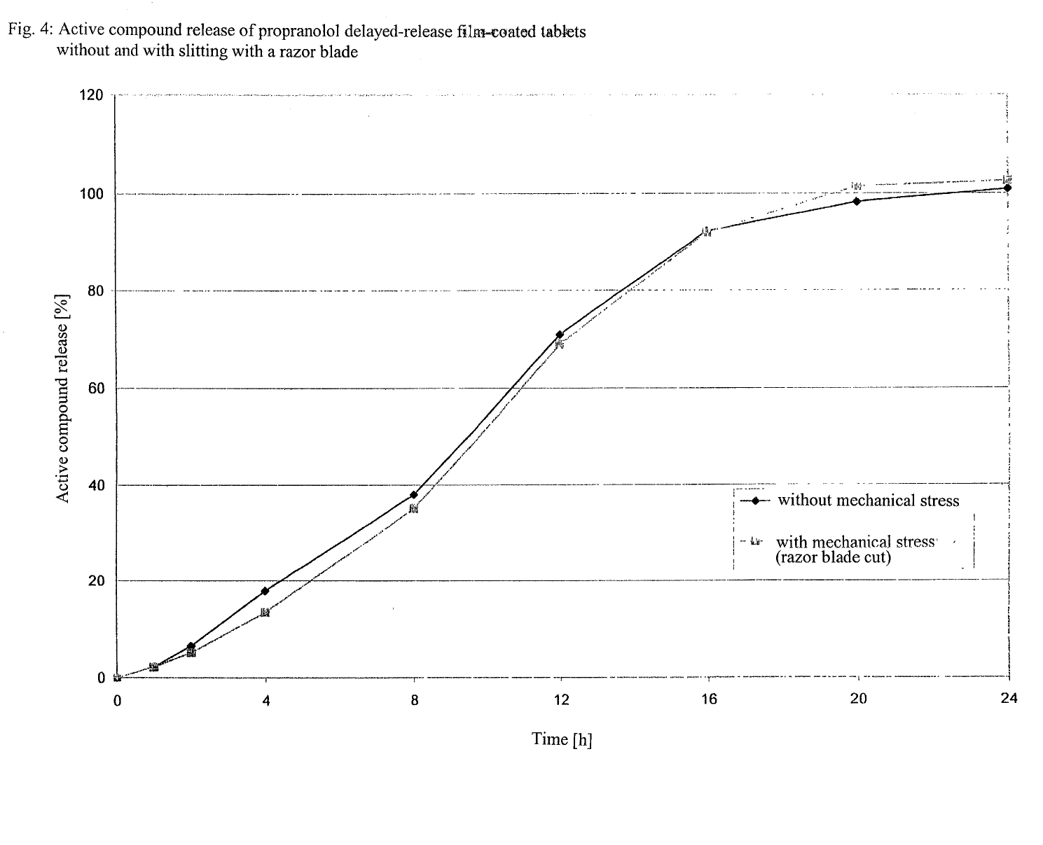

In FIG. 4 the active compound release of propranolol delayed-release film-coated tablets without and with slits using a razor blade is depicted.

### Example 4

### Pseudoephedrine Delayed-Release Film-Coated Tablet

5 kg of rapid-release pseudoephedrine tablets having a domed shape, a diameter of 9 mm and the following composition

| | |
|---|---|
| Pseudoephedrine-HCl | 100.0 mg |
| Ludipress LCE 8) | 150.0 mg |
| Kollidon VA 64 | 50.0 mg |
| Aerosil 200 | 3.0 mg |
| Magnesium stearate | 2.0 mg |
| Total weight | 305.0 mg |

are sprayed in a horizontal drum coater (Accela Cota 24″, Manesty) with the following coating preparation:

| Kollicoat SR 30 D | 43.5% (corresponds to 13.05% |
|---|---|
| (30% strength aqueous | of solid) |
| polyvinyl acetate | |
| dispersion) | |
| Triacetin | 1.3% |
| Kollicoat IR | 4.0% |
| Talc | 3.5% |
| Water | 47.7% |

The feed air temperature was 63° C., the product temperature 36° C. and the spray pressure 2.0 bar using a spray nozzle with a 1 mm opening width. The application rate was 16 mg/cm² corresponding to an amount of coating suspension of 2521 g. The coating thickness was 160 μm.

The coated tablets were released in 0.08 N HCl (0 to 2 h) and then in phosphate buffer pH 6.8 (2 to 24 h). Stressing by tipping 25 times from a height of 1 m onto a stone slab had no effects on the release.

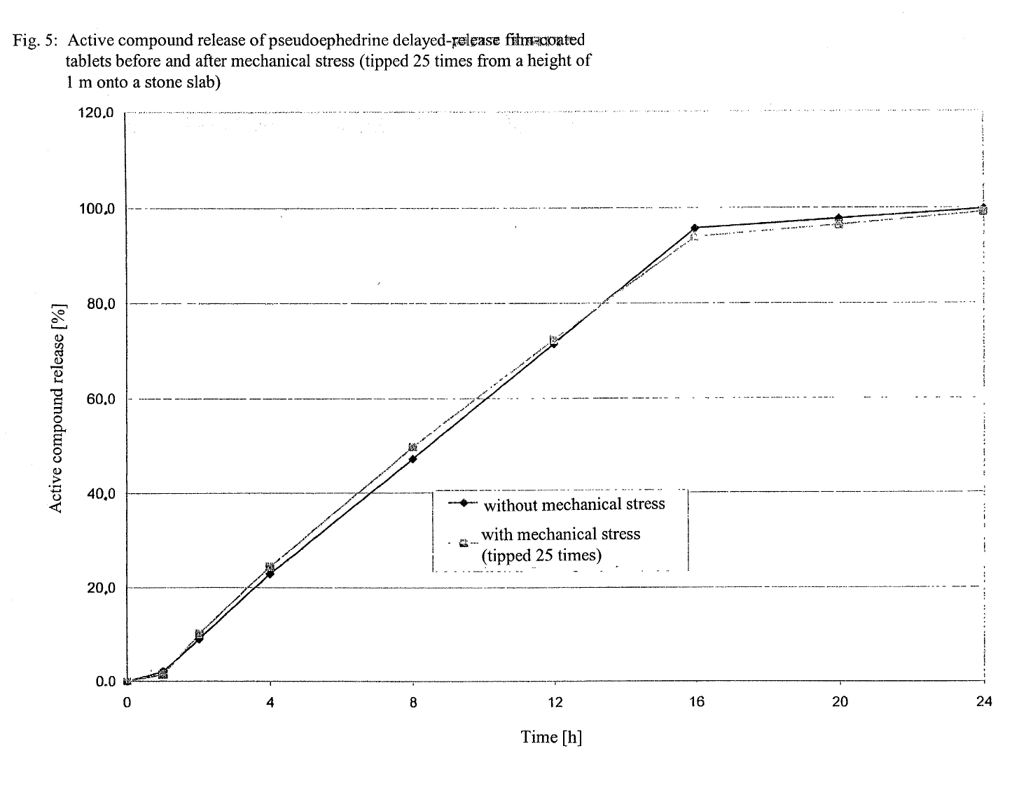

In FIG. 5 the active compound release of pseudoephedrine delayed-release film-coated tablets before and after mechanical stress (tipped 25 times from a height of 1 m onto a stone slab) is depicted.

### Example 5

### Metoprolol Delayed-Release Film-Coated Tablet with Additional Matrix Release Delaying

5 kg of slightly delayed-release metoprolol tablets having a domed shape, a diameter of 11 mm and the following composition

| | |
|---|---|
| Metoprolol tartrate | 200.0 mg |
| Kollidon SR 9) | 250.0 mg |
| Kollicoat SR 30 D | 6.0 mg |
| Magnesium stearate | 7.0 mg |
| Total weight | 463.0 mg |

are sprayed in a horizontal drum coater (Accela Cota 24″, Manesty) with the following coating preparation:

| Kollicoat SR 30 D | 40.0% (corresponds to 12.0% |
|---|---|
| 30% strength aqueous | of solid) |
| polyvinyl acetate | |
| dispersion) | |
| Kollicoat IR | 5.1% |
| Triacetin | 1.7% |
| Talc | 4.0% |
| Water | 49.2% |

The feed air temperature was 60° C., the product temperature 41° C. and the spray pressure 2.0 bar using a spray nozzle with a 1 mm opening width. The application rate was 16 mg/cm² corresponding to 2198 g. The layer thickness was 152 μm.

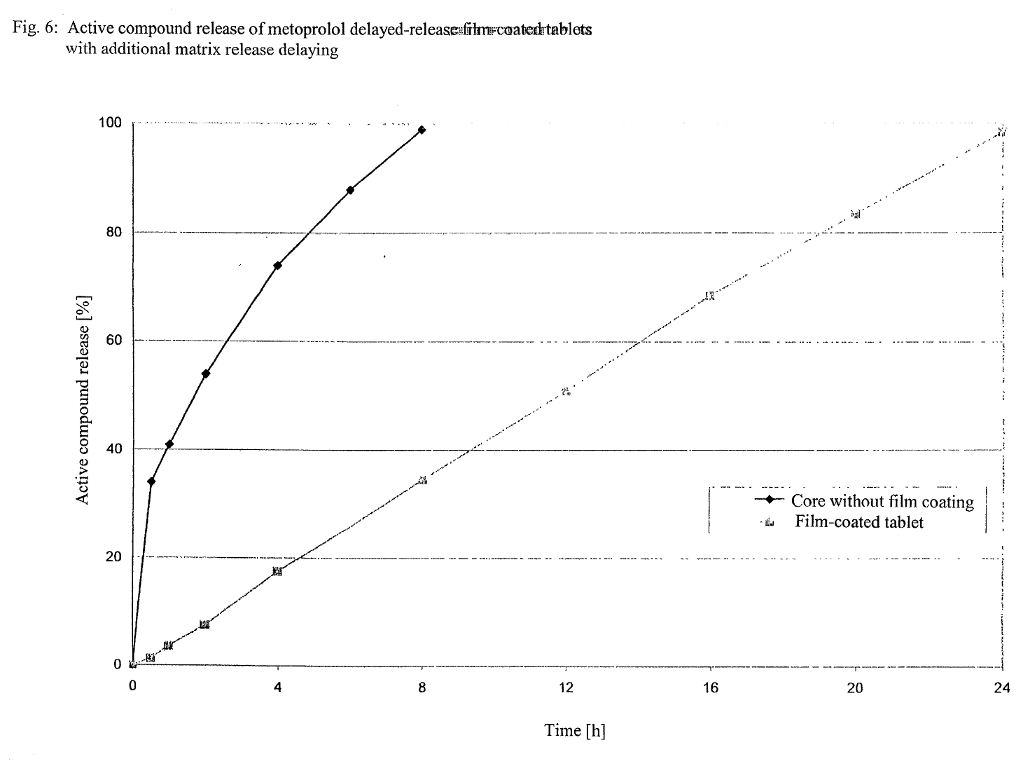

The coated tablets were initially released for 2 h in 0.08 N HCl and then in phosphate buffer pH 6.8, the active compound releases shown in FIG. 6 being achieved:

In FIG. 6: Active compound release of metoprolol delayed-release film-coated tablets with additional matrix release delaying

Neither pricking with a needle, slitting with a razor blade (cut length about 2 mm) nor mechanical stress in the Friabilator (1000 revolutions, falling height 15.5 cm) had any influence on the active compound release.

### Comparison Example

Analogously to example 3, propranolol tablets were coated with ethylcellulose dispersion (Aquacoat ECD, FMC) instead of polyvinyl acetate disperesion. In addition, the amount of plasticizer was doubled in order to achieve a better flexibility.

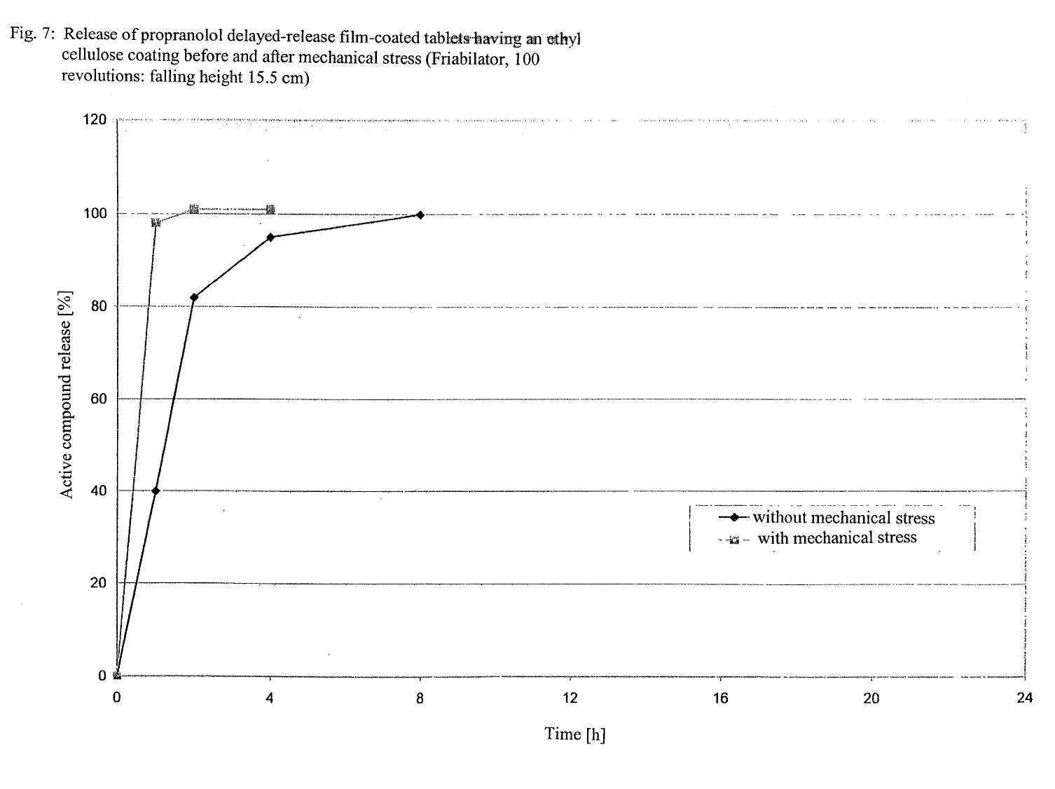

In FIG. 7 the release of propranolol delayed-release film-coated tablets with an ethylcellulose coating is depicted before and after mechanical stress (Friabilator, 100 revolutions, falling height 15.5 cm).

Coating with ethylcellulose produced no noticeable retardation and the active compound release was still considerably accelerated by mechanical stress. Distinct cracks could be detected in the coating after the mechanical stress. This shows that ethylcellulose is not suitable as a coating material for this purpose.

FIG. 3

| | without stress | pricked | friabilation test |
|---|---|---|---|
| 0 | 0.00 | 0.00 | 0.00 |
| 1 | 1.13 | 1.73 | 0.75 |
| 2 | 5.39 | 4.41 | 4.90 |
| 4 | 10.92 | 13.00 | 12.00 |
| 8 | 27.53 | 29.00 | 27.53 |
| 12 | 51.50 | 50.02 | 50.70 |
| 16 | 65.20 | 62.26 | 64.26 |
| 20 | 74.73 | 73.00 | 73.73 |
| 24 | 81.00 | 84.30 | 82.30 |

Metoprolol cores 6356

| | Di-Tab | Friabilator | defect |
|---|---|---|---|
| 0 | 0.00 | 0.00 | 0.00 |
| 2 | 12.04 | 13.24 | 10.84 |
| 5 | 18.65 | 20.52 | 16.79 |
| 10 | 25.63 | 28.19 | 23.07 |
| 15 | 31.29 | 34.42 | 28.16 |
| 30 | 43.87 | 48.26 | 39.48 |
| 60 | 58.54 | 64.39 | 52.68 |

FIG. 2

| | before | |
|---|---|---|
| | friabilation | friabilation |
| | test h | test |
| 0 | 0.00 | 0.00 |
| 1 | 3.53 | 4.83 |
| 2 | 11.73 | 13.13 |
| 4 | 34.63 | 34.63 |
| 8 | 64.10 | 62.10 |
| 12 | 82.02 | 81.00 |
| 16 | 91.93 | 93.83 |
| 20 | 100.00 | 100.00 |
| 24 | #NV | #NV |

FIG. 1

| | | (1a_ . . . | | | | |
|---|---|---|---|---|---|---|
| | without | | | | 10 | 16 |
| | coating | 4 mg/cm² | 6 mg/cm² | 8 mg/cm² | mg/cm² | mg/cm² |
| 0 | 0.00 | 0 | 0.00 | 0.00 | 0.00 | 0.00 |
| 1 | 100.00 | 18.19 | 7.21 | 3.53 | 2.16 | 1.00 |
| 2 | #NV | 37.96 | 19.30 | 11.73 | 8.08 | 2.20 |
| 4 | #NV | 64.18 | 44.22 | 34.63 | 27.15 | 6.40 |
| 8 | #NV | 87.63 | 74.10 | 64.10 | 56.35 | 22.00 |
| 12 | #NV | 97.56 | 89.77 | 82.02 | 76.07 | 38.00 |
| 16 | #NV | 100.17 | 96.41 | 91.83 | 87.02 | 52.00 |
| 20 | #NV | 100.17 | 100.0 | #NV | 93.65 | 63.75 |
| 24 | #NV | 100.17 | 100.0 | #NV | 100.17 | 77.00 |

FIG. 6

| | Core without | Film-coated |
|---|---|---|
| | film coating | tablet |
| 0 | 0 | 0 |
| 0.5 | 34 | 1.3 |
| 1 | 41 | 3.6 |
| 2 | 54 | 7.5 |
| 4 | 74 | 17.5 |
| 6 | 88 | #NV |
| 8 | 99 | 34.5 |
| 12 | #NV | 51.0 |
| 16 | #NV | 68.5 |
| 20 | #NV | 83.5 |
| 24 | #NV | 98.5 |

FIG. 7

| | Without | With |
|---|---|---|
| | mechanical | mechanical |
| 0 | 0 | 0 |
| 1 | 40 | 98.0 |
| 2 | 82 | 101.0 |
| 4 | 95 | 101.0 |
| 8 | 100 | #NV |
| 12 | #NV | #NV |
| 16 | #NV | #NV |
| 20 | #NV | #NV |
| 24 | #NV | #NV |

FIG. 4

| | Without | With mechanical stress |
|---|---|---|
| | mechanical | (razor blade cut) |
| 0 | 0 | 0 |
| 1 | 2.2 | 2.2 |
| 2 | 6.5 | 5.1 |
| 4 | 17.9 | 13.4 |
| 8 | 38.0 | 35.1 |
| 12 | 71.0 | 69.0 |
| 16 | 92.2 | 91.9 |
| 20 | 98.3 | 101.5 |
| 24 | 100.9 | 102.6 |

FIG. 5

| | Without | With mechanical stress |
|---|---|---|
| | mechanical | (tipped 25 times) |
| 0 | 0.0 | 0.0 |
| 1 | 2.1 | 1.5 |
| 2 | 9.0 | 10.1 |
| 4 | 23.0 | 24.5 |
| 8 | 47.3 | 49.8 |
| 12 | 71.6 | 72.5 |
| 16 | 95.8 | 94.0 |
| 20 | 97.8 | 96.5 |
| 24 | 99.8 | 99.1 |

We claim:

1. A pharmaceutica single unit administration form provided with a sealed film coating and having delayed release of active compound, the film coating containing 30 to 100% by weight of polyvinyl acetate as film-forming agent and the layer thickness of the film coating being 30 μm to 500 μm.

2. An administration form as claimed in claim 1, the film coating being applied from aqueous solution or suspension.

3. An administration form as claimed in claim 1 or 2, the polyvinyl acetate having a molecular weight of 10000 to 2000000 and being employed as an aqueous dispersion.

4. An administration form as claimed in one of claims 1 to 3, the film coating containing a water-insoluble polymer selected from the group consisting of ethylcellulose, methyl methacrylate-ethyl acrylate copolymers and ethyl acrylate-methyl methacrylate-trimethylammonium ethyl methacrylate chloride (ammonium methacrylate copolymer type A or B) in amounts of up to 40% by weight.

5. An administration form as claimed in one of claims 1 to 4, the film coating containing water-soluble polymers selected from the group consisting of polyvinyl alcohols, polyvinyl alcohol-polyethylene glycol graft copolymers, polyethylene glycols, ethylene oxide-propylene oxide block copolymers, alkylated celluloses, hydroxyalkylated celluloses, alkylated-hydroxyalkylated celluloses, carboxyalkylated celluloses, polyvinylpyrrolidones, vinylpyrrolidone-vinyl acetate copolymers, polyacrylates, polymethacrylates, acrylate-methacrylate copolymers, gelatin, polysaccharides, alginates, propylene glycol alginates, chitosans, carrageenans, gum arabic and their salts or derivatives in amounts from 5 to 30% by weight.

6. An administration form as claimed in one of claims 1 to 5, the film coating containing water-soluble substances selected from the group consisting of sugars, sugar alcohols, sugar derivatives, urea, and salts of pharmaceutically acceptable organic and inorganic acids in amounts from 5 to 30% by weight.

7. An administration form as claimed in one of claims 1 to 6, the film coating containing water-swellable substances selected from the group consisting of crosslinked polyvinylpyrrolidones, crosslinked polyacrylic acids, microcrystalline cellulose, starch, crosslinked sodium carboxymethylstarch, crosslinked sodium carboxymethylcellulose and pectins, in amounts from 5 to 30% by weight.

8. An administration form as claimed in one of claims 1 to 7, comprising substances customary for pharmaceutical film coatings, such as plasticizers, colorants, white pigments, fillers, antiadhesive agents, adhesion improvers, gloss intensifiers, surfactants and foam destroyers in amounts from 2 to 40% by weight.

9. An administration form as claimed in one of claims 1 to 8, 90% of the active compound being released in a period of time of 3 to 36 hours.

10. An administration form as claimed in one of claims 1 to 9, having a layer thickness of the film coating of 30 to 300 μm.

11. An administration form as claimed in one of claims 1 to 10 in form of tablets or capsules.

12. An administration form as claimed in one of claims 1 to 11, the coating consisting of two or more layers which differ with respect to their composition.

13. An administration form as claimed in one of claims 1 to 11, a further coating layer being applied under the polyvinyl acetate-containing film coating.

14. An administration form as claimed in one of claims 1 to 13, a further coating being applied to the polyvinyl acetate-containing layer.

15. An administration form as claimed in claim 14, the further coating layer being an enteric coating.

16. An administration form as claimed in one of claims 1 to 15, some of the active compound being incorporated into the coating layer.

17. An administration form as claimed in one of claims 1 to 16, a release-delaying agent being situated in the core.

18. An administration form as claimed in one of claims 1 to 17, pollyvinyl acetate being situated in the core.

19. An administration form as claimed in one of claims 1 to 18, the active compound being selected from the group consisting of of [sic] the groups human pharmaceuticals, veterinary pharmaceuticals, vitamins, carotenoids, nutraceuticals, food supplements, minerals and micronutrients.

20. A process for the production of administration forms as claimed in one of claims 1 to 19, which comprises applying a film-coating agent, containing polyvinyl acetate as film-forming agent, in amounts such that the resulting film coatings have a layer thickness of 30 to 500 μm.

21. A process as claimed in claim 20, wherein the administration forms are tempered at temperatures between 30 and 70° C. after application of the film coating.

22. A process as claimed in claim 20 or 21, wherein the layer thickness of the film coating is between 30 to [sic] 300 μm.

23. A process for the production of the administration form according to the invention as claimed in claim 1, wherein the aqueous coating preparation to be sprayed on has a solids content of 5 to 45% by weight.
